(19) United States
(12) Patent Application Publication
(10) Pub. No.: US 2024/0081366 A1
(43) Pub. Date: Mar. 14, 2024

(54) ORAL HYGIENE CHEWING GUM

(71) Applicant: Francene Di Bacco, Downsview (CA)

(72) Inventor: Francene Di Bacco, Downsview (CA)

(21) Appl. No.: 17/931,775

(22) Filed: Sep. 13, 2022

(51) Int. Cl. A23G 4/06; A23G 4/08; A61K 9/68

(52) U.S. Cl. CPC A23G 4/068; A23G 4/064; A23G 4/08; A61K 9/0058

(57) ABSTRACT

An oral hygiene chewing gum that is operable to provide a user detoxification and pH balancing of their mouth resulting in reduction of bacteria, control of halitosis and adsorption of harmful contaminants. The gum of the present invention is provided in a first embodiment and a second embodiment. Both the first embodiment and the second embodiment include a gum base, a sugar free sweetener and a flavoring agent. The first embodiment of the present invention is comprised of a composition that includes bentonite and charcoal. The second embodiment of the present invention includes a composition that further includes iodine and chlorophyll wherein the iodine and chlorophyll can be either liquid or powder when introduced into the manufacturing process. The first embodiment and the second embodiment of the present invention are both provided in unique flavors.

### FIELD OF THE INVENTION

The present invention relates generally to oral hygiene, more specifically but not by way of limitation, an oral hygiene gum having a first embodiment and a second embodiment wherein the oral hygiene gum of the present invention is operable to combat halitosis, absorb bacteria and yeast.

### BACKGROUND

Oral hygiene is an important part of overall healthcare. Proper brushing and flossing not only ensures the maintenance of healthy teeth but further reduces gum disease and other infections. As is known in the art, the common protocol for brushing teeth is the use of a toothbrush and a dentifrice wherein an amount of the dentifrice is placed on the toothbrush and the user will apply to their teeth utilizing various motions and pressure to accomplish the task of brushing the teeth. It is recommended that this procedure be executed at least twice a day and it is preferred by dental practitioners to be executed with even more frequency. The aforementioned protocol can also be accompanied by other tasks such as but not limited to flossing and utilization of an oral rinse.

One issue with conventional oral hygiene protocols is the requirement of equipment, time and a facility to perform the protocol. This results in most people engage in the aforementioned protocol only twice a day. However, people eat and drink during the day and the ingredients of the food and drink can be detrimental to the teeth and gums. It is not convenient to carry around tooth brushing implements and as such most people do not engage in an oral hygiene protocol during the day.

Accordingly, there is a need for an oral hygiene gum that can be chewed by a user wherein the gum is configured to provide oral hygiene care so as to offer an oral hygiene protocol intermediate brushing and flossing.

### SUMMARY OF THE INVENTION

It is the object of the present invention to provide an oral hygiene gum that is configured to provide oral hygiene to a user thereof wherein the present invention includes a first embodiment of gum and a second embodiment of gum.

Another object of the present invention is to provide a gum formulated with special ingredients to offer oral hygiene to a user thereof wherein the first embodiment of the gum of the present invention includes bentonite.

A further object of the present invention is to provide an oral hygiene gum that is configured to provide oral hygiene to a user thereof wherein the first embodiment of the gum further includes charcoal.

Still another object of the present invention is to provide a gum formulated with special ingredients to offer oral hygiene to a user thereof wherein the second embodiment of the gum of the present invention includes iodine.

An additional object of the present invention is to provide an oral hygiene gum that is configured to provide oral hygiene to a user thereof wherein the second embodiment of the gum of the present invention includes chlorophyll.

Yet a further object of the present invention is to provide a gum formulated with special ingredients to offer oral hygiene to a user thereof wherein both the first embodiment and second embodiment of the present invention include a gum base, a sugar free sweetener and a flavoring agent.

Another object of the present invention is to provide an oral hygiene gum that is configured to provide oral hygiene to a user thereof wherein both the first embodiment and second embodiment of the present invention are offered in unique flavors.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a chart of a composition of the first embodiment of the present invention; and

FIG. 2 is a chart of a composition of the second embodiment of the present invention.

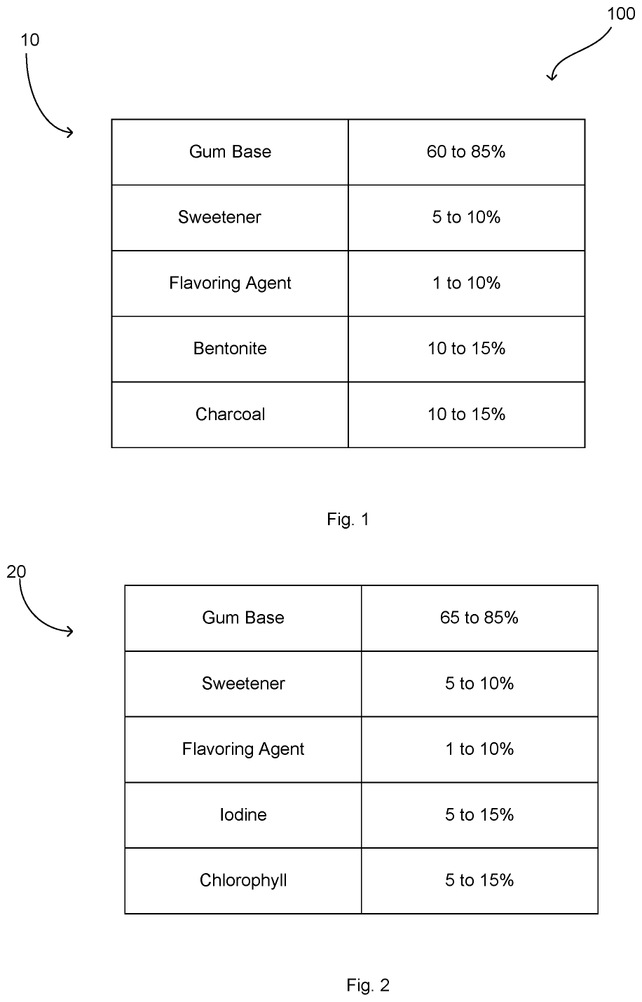

### DETAILED DESCRIPTION

Referring now to the drawings submitted herewith, wherein various elements depicted therein are not necessarily drawn to scale and wherein through the views and figures like elements are referenced with identical reference numerals, there is illustrated an oral hygiene chewing gum 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to Figures submitted as a part hereof, the oral hygiene chewing gum 100 is provided in a first embodiment 10 and a second embodiment 20 manufactured utilizing conventional techniques as is known in the art of chewing gum manufacture. Both the first embodiment and the second embodiment include a gum base, sugar free sweetener and a flavoring agent. While it is contemplated within the scope of the present invention that various alternate sugar free sweeteners could be utilized, in the preferred first embodiment and the second embodiment monk fruit or stevia is employed in the composition for the sugar free sweetener. For both the first embodiment and the second embodiment of the present invention the gum base is comprised of the same composition. As with conventional gums, the gum base of the present invention is non-nutritive, non-digestible, water-insoluble masticatory delivery system used to carry flavoring agents and the additional elements listed herein. The gum base of the present invention provides the basic textural and masticatory properties of gum providing a desirable chewing experience.

It is contemplated within the scope of the present invention that the gum base of the present invention is comprised of various combinations of the following synthetic coagulated or concentrated latices: Polymers such as butadiene-styrene, vinyl acetate and petroleum waxes are employed to create the gum base of the present invention. Additionally, plasticizing materials, softeners are utilized to emulsify components that may not bind to each other. Another component that can be employed as part of the gum base is terpene resin. It should be understood within the scope of the present invention that common preservatives constitute part of the gum base. Lastly, natural coagulated or concentrated latices of vegetable origin can be employed to create the gum base. These include many of the resins such as chicle and also includes natural waxes like beeswax and natural rubber.

The first embodiment of the present invention has a composition comprising gum base, sugar free sweetener, flavoring, bentonite and charcoal. In the preferred composition of the first embodiment of the oral hygiene chewing gum 100 the bentonite ingredient is the present in the amount of ten to fifteen percent of the total composition of the first embodiment of the present invention. The preferred composition of the first embodiment of the oral hygiene chewing gum 100 further includes charcoal wherein the charcoal comprises ten to fifteen percent of the composition of the first embodiment of the present invention. It is contemplated within the scope of the present invention that the charcoal could be charcoal or activated charcoal. While preferred percentages of the charcoal and bentonite have been disclosed herein, it is contemplated within the scope of the present invention that the alternate percentages of bentonite and charcoal could be provided within the ranges stated herein. Bentonite is an absorbent and is operable to absorb bacteria and yeast found naturally in the mouth which provides a technique of detoxification.

The oral hygiene chewing gum 100 includes a second embodiment 20. As with the first embodiment 10, the second embodiment 20 has a composition comprising gum base, sugar free sweetener and flavoring. The second embodiment 20 further includes iodine and chlorophyll. In a preferred composition of the second embodiment 20 of the oral hygiene chewing gum 100 iodine is present within a range of five to fifteen percent. Additionally, in a preferred composition of the second embodiment 20 of the present invention chlorophyll is included wherein the chlorophyll is present within the range of five to fifteen percent. It should be understood within the scope of the present invention that the chlorophyll and iodine could be obtained from either powdered or liquid forms so as to manufacture the second embodiment of the oral hygiene chewing gum 100. Iodine and chlorophyll are provided to balance the pH in a user's mouth which results in a lower level of cavity causing bacteria.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An oral hygiene chewing gum comprising:
a gum base,
a sugar free sweetener;
at least one flavoring agent;
a bentonite additive, wherein the bentonite additive comprises ten to fifteen percent of a total composition of the oral hygiene chewing gum; and
a charcoal additive, wherein the charcoal additive comprises ten to fifteen percent of the total composition of the oral hygiene chewing gum.

2. The oral hygiene chewing gum as recited in claim 1, wherein the charcoal additive can be activated charcoal.

3. The oral hygiene chewing gum as recited in claim 2, wherein the sugar free sweetener is a monk fruit sweetener.

4. The oral hygiene chewing gum as recited in claim 2, wherein the sugar free sweetener is stevia.

5. An oral hygiene chewing gum comprising:
a gum base,
a sugar free sweetener;
at least one flavoring agent;
a iodine additive, wherein the iodine additive comprises five to fifteen percent of a total composition of the oral hygiene chewing gum; and
a chlorophyll additive, wherein the chlorophyll additive comprises five to fifteen percent of the total composition of the oral hygiene chewing gum.

6. The oral hygiene chewing gum as recited in claim 5, wherein the chlorophyll is provided in a powdered form.

7. The oral hygiene chewing gum as recited in claim 6, wherein the iodine is provided in a powdered form.

8. The oral hygiene chewing gum as recited in claim 5, wherein the chlorophyll is provided in a liquid form.

9. The oral hygiene chewing gum as recited in claim 8, wherein the iodine is provided in a liquid form.
